(19) United States
(12) Patent Application Publication
(10) Pub. No.: US 2023/0386141 A1
(43) Pub. Date: Nov. 30, 2023

(54) METHOD, APPARATUS AND RECORDING MEDIUM STORING COMMANDS FOR PROCESSING SCANNED IMAGES OF 3D SCANNER

(71) Applicant: MEDIT CORP., Seoul (KR)

(72) Inventor: Young Mok CHO, Seoul (KR)

(73) Assignee: MEDIT CORP., Seoul (KR)

(21) Appl. No.: 18/323,161

(22) Filed: May 24, 2023

(30) Foreign Application Priority Data
May 27, 2022 (KR) 10-2022-0065542

(51) Int. Cl. G06T 19/00; G06V 10/764; G06T 7/00; G06V 10/82; G06T 7/11; A61B 1/24; A61B 1/00

(52) U.S. Cl. CPC G06T 19/00; G06V 10/764; G06T 7/0012; G06V 10/82; G06T 7/11; A61B 1/24; A61B 1/00172; A61B 1/00045; G06T 2207/20084; G06T 2207/10024; G06T 2207/20021; G06T 2200/24; G06T 2207/30036; G06V 2201/034; G06T 2207/20104; G06T 2210/41

(57) ABSTRACT

A method of processing scanned images of a 3D scanner is provided. The method is performed by an electronic apparatus, and includes: acquiring, from the 3D scanner, a 2D image set of an object generated by scan of the 3D scanner, the 2D image set including at least one 2D image; inputting an input image to an artificial neural network, which has been trained to detect at least one predetermined region in an image of the object, based on the 2D image set; detecting a first region in the input image based on an output of the artificial neural network; and generating 3D scan data of the object based on the first region.

### CROSS-REFERENCE TO RELATED APPLICATION

This application is based upon and claims the benefit of priority from Korean Patent Application No. 10-2022-0065542, filed on May 27, 2022, the entire contents of which are incorporated herein by reference.

### TECHNICAL FIELD

The present disclosure relates to a method for processing scanned images of a 3D scanner, and particularly, a method for detecting a specific region in an image received from a 3D scanner and generating 3D scan data based thereon.

### BACKGROUND

In general, in order to acquire oral cavity information of a patient, a 3D scanner that is inserted into the patient's oral cavity to acquire an image of the oral cavity may be used. For example, a doctor may insert a 3D scanner into the oral cavity of a patient and scan the patient's teeth, gingiva, and/or soft tissue, thereby acquiring a plurality of 2D images of the patient's oral cavity, and may construct a 3D image of the patient's oral cavity using the 2D images of the patient's oral cavity by applying 3D modeling technology.

At this time, in the case of patients undergoing treatment, orthodontic devices such as wires and prostheses, and artificial teeth such as crowns may be present in the oral cavity.

Conventionally, in order to acquire such oral cavity information of a patient, a 3D image was constructed by scanning the oral cavity of the patient after removing an orthodontic device. However, this has a problem of not only increasing the time required to obtain the 3D image, but also making it difficult to obtain a proper 3D image if the orthodontic device cannot be removed because the patient's oral cavity is scanned with the orthodontic device present. Therefore, in the art, there has been an increasing demand for a technique for more accurately scanning a 3D image of a patient's oral cavity even when a device for treatment is present in the patient's oral cavity.

### SUMMARY

Various embodiments of the present disclosure provide a method of detecting a specific region in an image received from a 3D scanner and generating 3D scan data based thereon.

According to various embodiments of the present disclosure, an artificial neural network is used to detect a specific region within a scanned image and 3D scan data of an object is generated based thereon. Therefore, a user can generate the 3D scan data in which the specific regions is not represented. Accordingly, unnecessary information can be excluded from the 3D scan data for examination or treatment, and necessary information can be provided to the user more accurately and concisely. Furthermore, if a plurality of specific regions included in the 3D scan data are visually differentiated to the user, the user can quickly and conveniently distinguish between different specific regions included in the 3D scan data.

According to one aspect of the present disclosure, there is provided a method of processing scanned images of a 3D scanner. The method may be performed by an electronic apparatus, and include: acquiring, from the 3D scanner, a 2D image set of an object generated by scan of the 3D scanner, the 2D image set including at least one 2D image; inputting an input image to an artificial neural network, which has been trained to detect at least one predetermined region in an image of the object, based on the 2D image set; detecting a first region in the input image based on an output of the artificial neural network; and generating 3D scan data of the object based on the first region.

In one embodiment, the first region may be a region corresponding to metal in the input image.

In one embodiment, the 2D image set may include: at least one 2D image acquired by irradiating the object with patterned light through the 3D scanner; and at least one 2D image acquired by irradiating the object with non-patterned light through the 3D scanner.

In one embodiment, the input image input to the artificial neural network may be generated based on at least one 2D image acquired by irradiating the object with non-patterned light.

In one embodiment, inputting the input image to the artificial neural network may include: generating an red-green-blue (RGB) image using two or more 2D images, which are included in the 2D image set and used to acquire monochrome information; and inputting the RGB image to the artificial neural network.

In one embodiment, the artificial neural network may have been trained to output a segmentation result for the input image by classifying at least one pixel included in the input image into a corresponding region among the at least one predetermined region.

In one embodiment, generating the 3D scan data of the object may include generating the 3D scan data based on the 2D image set such that the coordinates corresponding to the first region are not included in the 3D scan data.

In one embodiment, generating the 3D scan data based on the 2D image set such that the coordinates corresponding to the first region are not included in the 3D scan data may include: excluding values of pixels corresponding to the first region in each 2D image included in the 2D image set by from a calculation target.

In one embodiment, generating the 3D scan data of the object may include: removing data of a region corresponding to the first region from the at least one 2D image included in the 2D image set; and generating the 3D scan data using the at least one 2D image from which the data of the region corresponding to the first region are removed.

In one embodiment, removing the data of the region corresponding to the first region from the at least one 2D image included in the 2D image set may include changing values of pixels included in the region corresponding to the first region in the at least one 2D image to a preset value.

In one embodiment, detecting the first region may include detecting a plurality of different first regions in the input image based on the output of the artificial neural network, and generating the 3D scan data may include generating the 3D scan data such that the plurality of first regions are distinguished from each other.

In one embodiment, the method may further include: acquiring user input on whether the first region is to be included, and generating the 3D scan data may include determining whether the coordinates corresponding to the first region are included in the 3D scan data according to the user input.

According to another aspect of the present disclosure, there is provided an electronic apparatus for processing scanned images of a 3D scanner. The electronic apparatus may include: a communication circuit communicatively connected to a 3D scanner; a memory; a display; and one or more processors. The one or more processors may be configured to: acquire, from the 3D scanner, a 2D image set of an object generated by scan of the 3D scanner, the 2D image set including at least one 2D image; input an input image to an artificial neural network, which has been trained to detect at least one predetermined region in an image of the object, based on the 2D image set; detect a first region in the input image based on an output of the artificial neural network; and generate 3D scan data of the object based on the first region.

In one embodiment, the one or more processors may be configured to: generate an RGB image using two or more 2D images included in the 2D image set and used to acquire monochrome information; and input the RGB image to the artificial neural network.

In one embodiment, the artificial neural network may have been trained to output a segmentation result for the input image by classifying at least one pixel included in the input image into a corresponding region among the at least one predetermined region.

In one embodiment, the one or more processors may be configured to generate the 3D scan data based on the 2D image set such that the coordinates corresponding to the first region are not included in the 3D scan data.

In one embodiment, the one or more processors may be configured to exclude values of pixels corresponding to the first region in each 2D image included in the 2D image set from a calculation target.

In one embodiment, the one or more processors may be configured to: remove data of a region corresponding to the first region from the at least one 2D image included in the 2D image set; and generate the 3D scan data using the at least one 2D image from which the data of the region corresponding to the first region are removed.

In one embodiment, the one or more processors may be configured to change values of pixels included in the region corresponding to the first region in the at least one 2D image to a preset value.

In one embodiment, the one or more processors may be configured to: detect a plurality of different first regions in the input image based on the output of the artificial neural network; and generate the 3D scan data such that the plurality of first regions are distinguished from each other.

According to another aspect of the present disclosure, there is provided a non-transitory computer-readable recording medium storing instructions for processing scanned images of a 3D scanner, which are performed on a computer. When executed by one or more processors, the instructions cause the one or more processors to: acquire, from a 3D scanner, a 2D image set of an object generated by scan of the 3D scanner, the 2D image set including at least one 2D image; input an input image to an artificial neural network, which has been trained to detect at least one predetermined region in an image of the object, based on the 2D image set; detect a first region in the input image based on an output of the artificial neural network; and generate 3D scan data of the object based on the first region.

### BRIEF DESCRIPTION OF DRAWINGS

The accompanying drawings, which are incorporated in and constitute a part of the specification, illustrate embodiments of the present disclosure.

FIG. 1 is a view showing acquisition of an image of a patient's oral cavity using a 3D scanner according to an embodiment of the present disclosure.

FIG. 2A is a block diagram of an electronic apparatus and a 3D scanner according to an embodiment of the present disclosure.

FIG. 2B is a perspective view of a 3D scanner according to an embodiment of the present disclosure.

FIG. 3 is a view showing an example of a 2D image set and 3D scan data according to an embodiment of the present disclosure.

FIG. 4 is an operational flowchart of an electronic apparatus according to an embodiment of the present disclosure.

FIG. 5 is an exemplary view showing images included in a 2D image set according to an embodiment of the present disclosure.

FIG. 6 is a conceptual diagram showing input/output data of an artificial neural network according to an embodiment of the present disclosure.

FIG. 7 is a view illustrating input/output data of the artificial neural network according to an embodiment of the present disclosure.

FIG. 8 is a view illustrating 3D scan data according to an embodiment of the present disclosure.

FIG. 9 is an exemplary view visually representing 3D scan data generated to distinguish a plurality of first regions from each other according to an embodiment of the present disclosure.

FIG. 10 is an exemplary diagram showing a user interface screen for receiving user input regarding whether or not a specific region is included in the 3D scan data.

### DETAILED DESCRIPTION

Embodiments of the present disclosure are illustrated for the purpose of explaining the technical ideas of the present disclosure. The scope of claims in accordance with the present disclosure is not limited to the following embodiments and the specific description of these embodiments.

All technical or scientific terms used herein have meanings that are generally understood by a person having ordinary knowledge in the art to which the present disclosure pertains, unless otherwise specified. The terms used herein are selected for more clear illustration of the present disclosure, and are not intended to limit the scope of claims in accordance with the present disclosure.

The expressions “include,” “provided with,” “have” and the like used herein should be understood as open-ended terms connoting the possibility of inclusion of other embodiments, unless otherwise mentioned in a phrase or sentence including the expressions.

A singular expression can include meanings of plurality unless otherwise mentioned, and the same is applied to a singular expression stated in the claims. The terms “first,” “second,” etc. used herein are used to identify a plurality of components from one another, and are not intended to limit the order or importance of the relevant components.

The term “unit” used in these embodiments means a software component or hardware component, such as a field-programmable gate array (FPGA) and an application specific integrated circuit (ASIC). However, a “unit” is not limited to software and hardware. It may be configured to be an addressable storage medium or may be configured to run on one or more processors. For example, a “unit” may include components, such as software components, object-oriented software components, class components, and task components, as well as processors, functions, attributes, procedures, subroutines, segments of program codes, drivers, firmware, micro-codes, circuits, data, databases, data structures, tables, arrays, and variables. Functions provided in components and “unit” may be combined into a smaller number of components and “units” or further subdivided into additional components and “units.”

The expression “based on” used herein is used to describe one or more factors that influence a decision, an action of judgment or an operation described in a phrase or sentence including the relevant expression, and this expression does not exclude an additional factor influencing the decision, the action of judgment or the operation.

When a certain component is described as “coupled to” or “connected to” another component, this should be understood as having a meaning that the certain component may be coupled or connected directly to the other component or that the certain component may be coupled or connected to the other component via a new intervening component.

In the present disclosure, artificial intelligence (AI) refers to a technology that imitates human learning ability, reasoning ability, and perception ability and implements them with a computer, and may include the concepts of machine learning and symbolic logic. The machine learning (ML) may be an algorithm technology that classifies or learns features of input data by itself. Artificial intelligence technology is a machine learning algorithm that can analyze input data, learn the result of the analysis, and make judgments or predictions based on the result of the learning. In addition, technologies that use the machine learning algorithm to imitate the cognitive and judgmental functions of the human brain can also be understood as a category of artificial intelligence. For example, technical fields of linguistic understanding, visual understanding, inference/prediction, knowledge expression, and motion control may be included in the category of artificial intelligence.

In the present disclosure, the machine learning may refer to a process of training a neural network model using experience of processing data. Through the machine learning, computer software may mean improving its own data processing capabilities. The neural network model is constructed by modeling the correlation between data, and the correlation may be expressed by a plurality of parameters. The neural network model may derive the correlation between data by extracting and analyzing features from given data, and optimizing the parameters of the neural network model by repeating this process may be referred to as machine learning. For example, the neural network model may learn mapping (correlation) between an input and an output with respect to data given as an input/output pair. Alternatively, even when only input data are given, the neural network model may learn the relationship by deriving the regularity between given data.

In the present disclosure, an artificial neural network, an artificial intelligence learning model, a machine learning model, or a neural network model may be designed to implement a human brain structure on a computer, and may include a plurality of network nodes that simulate neurons of a human neural network and have weights. The plurality of network nodes may have a connection relationship between them by simulating synaptic activities of neurons that exchange signals through synapses. In the artificial neural network, a plurality of network nodes may exchange data according to a convolution connection relationship while being located in layers of different depths. The artificial neural network may be, for example, an artificial neural network model, a convolution neural network model, or the like. Hereinafter, embodiments of the present disclosure will be described with reference to the accompanying drawings. In the accompanying drawings, like or relevant components are indicated by like reference numerals. In the following description of embodiments, repeated descriptions of the identical or relevant components will be omitted. However, even if a description of a component is omitted, such a component is not intended to be excluded in an embodiment.

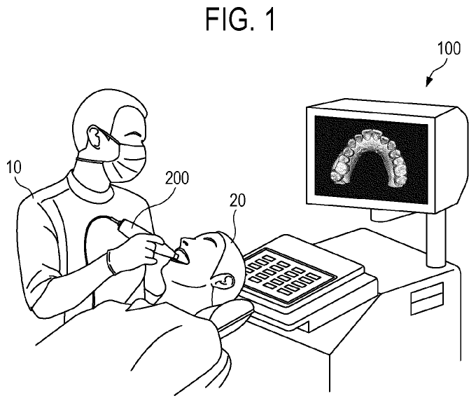

FIG. 1 is a view showing acquisition of an image of a patient's oral cavity using a 3D scanner 200 according to an embodiment of the present disclosure.

According to various embodiments, the 3D scanner 200 may be a dental medical device for acquiring an image of the oral cavity of an object 20. For example, the 3D scanner 200 may be an intraoral scanner. As shown in FIG. 1, a user 10 (e.g., a dentist or a dental hygienist) may acquire an image of the oral cavity of the object 20 (e.g., a patient) from the object 20 using the 3D scanner 200. As another example, the user 10 may also acquire an image of the oral cavity of the object 20 from a diagnosis model (e.g., a plaster model or an impression model) imitating the shape of the oral cavity of the object 20. Hereinafter, for convenience of explanation, it will be described that an image of the oral cavity of the object 20 is acquired by scanning the oral cavity of the object 20, but without being limited thereto, an image of other parts (e.g., ears) of the object 20 may be acquired. The 3D scanner 200 may have a form capable of insertion into and withdrawal from the oral cavity, and may be a handheld scanner in which the user 10 can freely adjust a scanning distance and a scanning angle.

The 3D scanner 200 according to various embodiments may acquire an image of the oral cavity by being inserted into the oral cavity of the object 20 and scanning the oral cavity in a non-contact manner. The image of the oral cavity may include at least one tooth, gingiva, and artificial structure insertable into the oral cavity (e.g., an orthodontic device including a bracket and a wire, an implant, a denture, and an orthodontic aid). The 3D scanner 200 may irradiate the oral cavity of the object 20 (e.g., at least one tooth or gingiva of the object 20) with light using a light source (or a projector) and may receive light reflected from the oral cavity of the object 20 through a camera (or at least one image sensor). According to another embodiment, the 3D scanner 200 may acquire an image of a diagnosis model of the oral cavity by scanning the diagnosis model of the oral cavity. When the diagnosis model of the oral cavity is a diagnosis model that imitates the shape of the oral cavity of the object 20, the image of the diagnosis model of the oral cavity may be an image of the oral cavity of the object. Hereinafter, for convenience of explanation, a case in which an image of the oral cavity is acquired by scanning the inside of the oral cavity of the object 20 is assumed, but is not limited thereto.

The 3D scanner 200 according to various embodiments may acquire a surface image of the oral cavity of the object 20 as a 2D image based on information received through a camera. The surface image of the oral cavity of the object 20 may include at least one among at least one tooth, gingiva, artificial structure, and cheek, tongue, or lip of the object 20. The surface image of the oral cavity of the object 20 may be a 2D image.

The 2D image of the oral cavity acquired by the 3D scanner 200 according to various embodiments may be transmitted to an electronic apparatus 100 connected through a wired or wireless communication network. The electronic apparatus 100 may be a computer apparatus or a portable communication apparatus. The electronic apparatus 100 may generate a 3D image of the oral cavity (or a 3D oral cavity image or a 3D oral cavity model) representing the oral cavity in 3D based on the 2D image of the oral cavity received from the 3D scanner 200. The electronic apparatus 100 may generate a 3D image of the oral cavity by 3D-modeling the internal structure of the oral cavity based on the received 2D image of the oral cavity.

The 3D scanner 200 according to yet another embodiment may acquire a 2D image of the oral cavity by scanning the oral cavity of the object 20, generate a 3D image of the oral cavity based on the obtained 2D image of the oral cavity, and transmit the generated 3D image of the oral cavity to the electronic apparatus 100.

The electronic apparatus 100 according to various embodiments may be communicatively connected to a cloud server (not shown). In this case, the electronic apparatus 100 may transmit a 2D image or 3D image of the oral cavity of the object 20 to the cloud server, and the cloud server may store the 2D image or 3D image of the oral cavity of the object 20 received from the electronic apparatus 100.

According to still another embodiment, as the 3D scanner, a table scanner (not shown) fixed to a specific position may be used in addition to the handheld scanner inserted into the oral cavity of the object 20. The table scanner may generate a 3D image of the diagnosis model of the oral cavity by scanning the diagnosis model of the oral cavity. In this case, since a light source (or a projector) and a camera of the table scanner are fixed, a user can scan the diagnosis model of the oral cavity while moving the diagnosis model of the oral cavity.

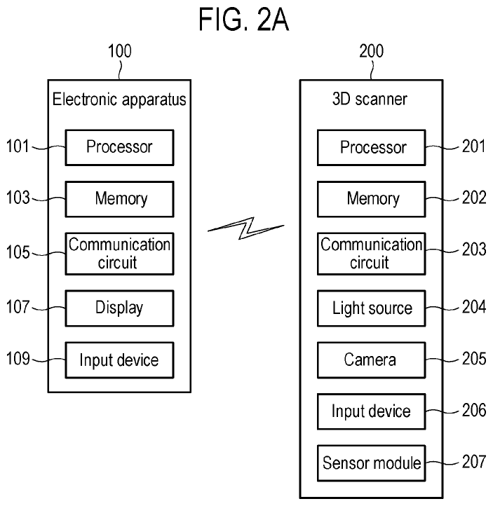

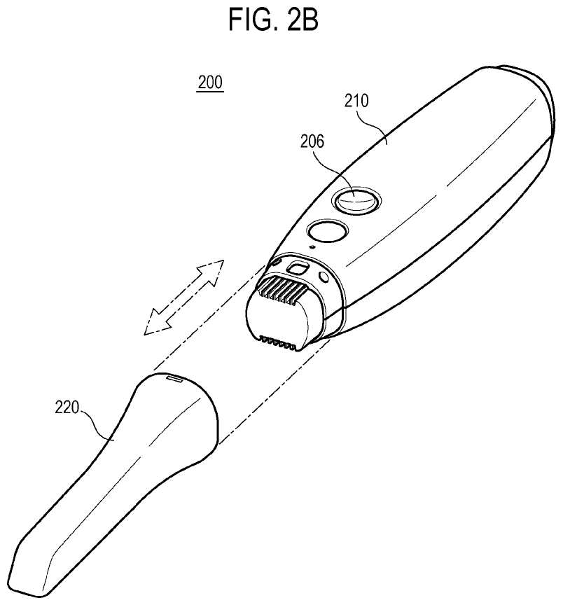

FIG. 2A is a block diagram of the electronic apparatus 100 and the 3D scanner 200 according to an embodiment of the present disclosure.

The electronic apparatus 100 and the 3D scanner 200 may be communicatively connected to each other through a wired or wireless communication network and may transmit/receive various data to/from each other.

The 3D scanner 200 according to various embodiments may include a processor 201, a memory 202, a communication circuit 203, a light source 204, a camera 205, an input device 206, and/or a sensor module 207. At least one of the components included in the 3D scanner 200 may be omitted or another component may be added to the 3D scanner 200. Additionally or alternatively, some of the components may be integrated, or may be implemented as a single entity or a plurality of entities. At least some of the components in the 3D scanner 200 may be connected to each other through a bus, a general purpose input/output (GPIO), a serial peripheral interface (SPI), a mobile industry processor interface (MIPI), or the like, to exchange data and/or signals.

The processor 201 of the 3D scanner 200 according to various embodiments, which is a component that can perform calculation or data processing related to control and/or communication of each component of the 3D scanner 200, may be operatively connected to the other components of the 3D scanner 200. The processor 201 may load commands or data received from the other components of the 3D scanner 200 into the memory 202, process the commands or data stored in the memory 202, and store the resultant data. The memory 202 of the 3D scanner 200 according to various embodiments may store instructions for the operation of the processor 201.

According to various embodiments, the communication circuit 203 of the 3D scanner 200 may establish a wired or wireless communication channel with an external apparatus (e.g., the electronic apparatus 100) and transmit/receive various data to/from the external apparatus. According to one embodiment, the communication circuit 203 may include at least one port connected to the external apparatus through a wired cable in order to communicate with the external apparatus by wire. In this case, the communication circuit 203 may perform communication with the external apparatus connected by wire through at least one port. According to one embodiment, the communication circuit 203 may be configured to be connected to a cellular network (e.g., 3G, LTE, 5G, Wibro, or Wimax) including a cellular communication module. According to various embodiments, the communication circuit 203 may transmit/receive data to/from the external apparatus by using short-range communication (e.g., Wi-Fi, Bluetooth, Bluetooth Low Energy (BLE), or UWB) including a short-range communication module, but is not limited thereto. According to one embodiment, the communication circuit 203 may include a non-contact communication module for non-contact communication. The non-contact communication may include, for example, at least one non-contact type proximity communication technology such as near field communication (NFC), radio frequency identification (RFID) communication, or magnetic secure transmission (MST) communication.

The light source 204 of the 3D scanner 200 according to various embodiments may irradiate the oral cavity of the object 20 with light. For example, the light emitted from the light source 204 may be structured light having a predetermined pattern (e.g., a stripe pattern in which straight line patterns of different colors continuously appear). The structured light pattern may be generated using, for example, a pattern mask or a digital micro-mirror device (DMD), but is not limited thereto. The camera 205 of the 3D scanner 200 according to various embodiments may acquire an image of the oral cavity of the object 20 by receiving reflected light reflected by the oral cavity of the object 20. The camera 205 may include, for example, a left camera corresponding to the left eye field of view and a right camera corresponding to the right eye field of view in order to build a 3D image according to an optical triangulation method. The camera 205 may include at least one image sensor such as a CCD sensor or a CMOS sensor. The input device 206 of the 3D scanner 200 according to various embodiments may receive user input for controlling the 3D scanner 200. The input device 206 may include a button for receiving push manipulation of the user 10, a touch panel for detecting touch of the user 10, and a voice recognition device including a microphone. For example, the user 10 may control starting or stopping of scanning using the input device 206.

The sensor module 207 of the 3D scanner 200 according to various embodiments may detect an operating state of the 3D scanner 200 or an external environmental state (e.g., user's motion) and generate an electrical signal corresponding the detected state. The sensor module 207 may include, for example, at least one of a gyro sensor, an acceleration sensor, a gesture sensor, a proximity sensor, and an infrared sensor. The user 10 may control starting or stopping of scanning using the sensor module 207. For example, if the user 10 moves with the 3D scanner 200 held in the user's hand, the 3D scanner 200 may control the processor 201 to start a scanning operation when an angular velocity measured through the sensor module 207 exceeds a preset threshold value.

According to one embodiment, the 3D scanner 200 may receive user input for starting the scan through the input device 206 of the 3D scanner 200 or the input device 206 of the electronic apparatus 100, or may start scanning according to a process by the processor 201 of the 3D scanner 200 or the processor 201 of the electronic apparatus 100. When the user 10 scans the inside of the oral cavity of the object 20 through the 3D scanner 200, the 3D scanner 200 may generate a 2D image of the oral cavity of the object 20, and in real time, may transmit the 2D image of the oral cavity of the object 20 to the electronic apparatus 100. The electronic apparatus 100 may display the received 2D image of the oral cavity of the object 20 on a display. Further, the electronic apparatus 100 may generate (build) a 3D image of the oral cavity of the object 20 based on the 2D image of the oral cavity of the object 20, and may display the 3D image of the oral cavity on the display. The electronic apparatus 100 may also display the 3D image being generated, on the display in real time.

The electronic apparatus 100 according to various embodiments may include one or more processors 101, one or more memories 103, a communication circuit 105, a display 107, and/or an input device 109. At least one of the components included in the electronic apparatus 100 may be omitted or another component may be added to the electronic apparatus 100. Additionally or alternatively, some of the components may be integrated, or may be implemented as a single entity or a plurality of entities. At least some of the components in the electronic apparatus 100 are connected to each other through a bus, a general purpose input/output (GPIO), a serial peripheral interface (SPI), a mobile industry processor interface (MIPI), or the like, to exchange data and/or signals.

According to various embodiments, the one or more processors 101 of the electronic apparatus 100 may be a component that can perform calculation or data processing related to control and/or communication of each component (e.g., the memory 103) of the electronic apparatus 100. The one or more processors 101 may be operatively connected to the other components of the electronic apparatus 100, for example. The one or more processors 101 may load commands or data received from the other components of the electronic apparatus 100 into the one or more memories 103, process the commands or data stored in the one or more memories 103, and store the resulting data.

According to various embodiments, the one or more memories 103 of the electronic apparatus 100 may store instructions for the operation of the one or more processors 101. The one or more memories 103 may store correlation models built according to a machine learning algorithm. The one or more memories 103 may store data (e.g., a 2D image of the oral cavity acquired through oral cavity scan) received from the 3D scanner 200.

According to various embodiments, the communication circuit 105 of the electronic apparatus 100 may establish a wired or wireless communication channel with an external apparatus (e.g., the 3D scanner 200, a cloud server, etc.), and transmit/receive various data with the external apparatus. According to one embodiment, the communication circuit 105 may include at least one port connected to the external apparatus through a wired cable in order to communicate with the external apparatus by wire. In this case, the communication circuit 105 may perform communication with the external apparatus connected by wire through the at least one port. According to one embodiment, the communication circuit 105 may be configured to be connected to a cellular network (e.g., 3G, LTE, 5G, Wibro, or Wimax) including a cellular communication module. According to various embodiments, the communication circuit 105 may transmit/receive data to/from the external apparatus by using short-range communication (e.g., Wi-Fi, Bluetooth, Bluetooth Low Energy (BLE), or UWB) including a short-range communication module, but is not limited thereto. According to one embodiment, the communication circuit 105 may include a non-contact communication module for non-contact communication. The non-contact communication may include, for example, at least one non-contact type proximity communication technology such as near field communication (NFC), radio frequency identification (RFID) communication, or magnetic secure transmission (MST) communication.

The display 107 of the electronic apparatus 100 according to various embodiments may display various screens based on the control of the processor 101. The processor 101 may display a 2D image of the oral cavity of the object 20 received from the 3D scanner 200 and/or a 3D image of the oral cavity in which the internal structure of the oral cavity is 3D-modeled, on the display 107. For example, the 2D image and/or the 3D image of the oral cavity may be displayed through a specific application program. In this case, the user 10 can edit, save, and delete the 2D image and/or the 3D image of the oral cavity.

The input device 109 of the electronic apparatus 100 according to various embodiments may receive commands or data to be used in a component (e.g., the one or more processors 101) of the electronic apparatus 100 from the outside (e.g., a user). The input device 109 may include, for example, a microphone, a mouse, or a keyboard. According to one embodiment, the input device 109 may be implemented in the form of a touch sensor panel capable of recognizing contact or proximity of various external objects by being combined with the display 107.

FIG. 2B is a perspective view of the 3D scanner 200 according to an embodiment of the present disclosure.

The 3D scanner 200 according to various embodiments may include a main body 210 and a probe tip 220. The main body 210 of the 3D scanner 200 may be formed in a shape that is easy for the user 10 to grip with hand. The probe tip 220 may be formed in a shape that facilitates insertion into and withdrawal from the oral cavity of the object 20. In addition, the main body 210 may be combined with and separated from the probe tip 220. The components of the 3D scanner 200 described in FIG. 2A may be disposed inside the main body 210. An opening opened so as to irradiate the object 20 with light output from the light source 204 may be formed at one end of the main body 210. The light output through the opening may be reflected by the object 20 and introduced again through the opening. The reflected light introduced through the opening may be captured by a camera to generate an image of the object 20. The user 10 may start scanning using the input device 206 (e.g., a button) of the 3D scanner 200. For example, when the user 10 touches or presses the input device 206, the object 20 may be radiated with the light from the light source 204.

In one embodiment, the user 10 may scan the inside of the oral cavity of the object 20 while moving the 3D scanner 200, in which case, the 3D scanner 200 may acquire at least one 2D image of the oral cavity of the object 20. For example, the 3D scanner 200 may acquire a 2D image of a region including incisors of the object 20 and a 2D image of a region including molar teeth of the object 20. The 3D scanner 200 may transmit the acquired at least one 2D image to the electronic apparatus 100.

According to another embodiment, the user 10 may scan a diagnosis model while moving the 3D scanner 200, and may acquire at least one 2D image of the diagnosis model in the process. Hereinafter, for convenience of explanation, a case in which an image of the oral cavity of the object 20 is acquired by scanning the inside of the oral cavity of the object 20 is assumed, but is not limited thereto.

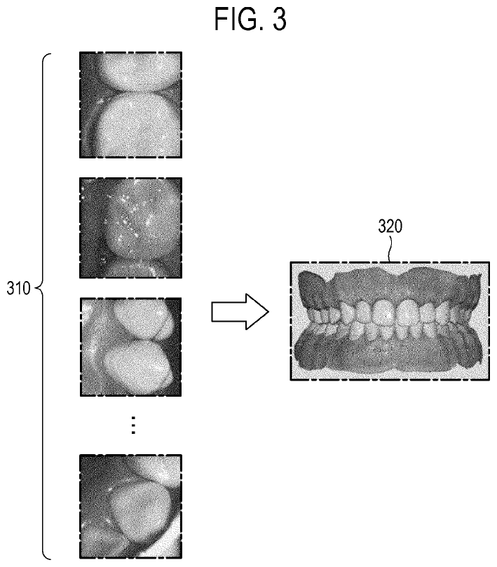

FIG. 3 is an exemplary view showing a 2D image set and 3D scan data according to an embodiment of the present disclosure.

The electronic apparatus 100 according to an embodiment of the present disclosure may acquire a 2D image set 310 including at least one 2D image by scan of the 3D scanner 200, and generate 3D scan data 320 of the object 20 based on the acquired 2D image set 310. The 3D scan data 320 may be data that is expressed on a 3D coordinate plane, and may include a plurality of 3D coordinate values. For example, the electronic apparatus 100 may generate a point cloud data set, which is a set of data points having 3D coordinate values, as the 3D scan data 320. The electronic apparatus 100 may generate 3D scan data including a smaller number of data points by aligning the point cloud data set. The electronic apparatus 100 may generate 3D scan data updated by reconstructing (rebuilding) the 3D scan data. For example, the electronic apparatus 100 may merge at least some data of the 3D scan data stored as raw data using a Poisson algorithm, and reconstruct a plurality of data points to reconstruct the 3D scan data so that the data points included in the 3D scan data can be a closed 3D surface when they are visually represented.

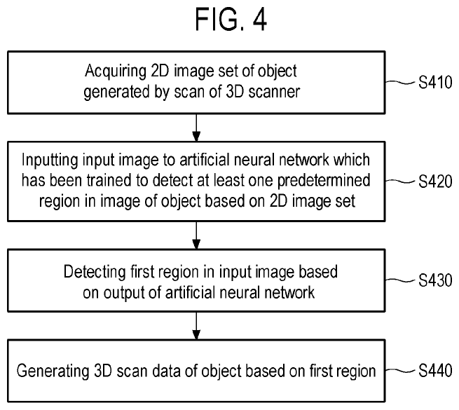

FIG. 4 is an operational flowchart of the electronic apparatus according to an embodiment of the present disclosure.

In step S410, the processor 101 may acquire a 2D image set of the object 20 generated by scan of the 3D scanner, from the 3D scanner 200. The 2D image set may include at least one 2D image. In the present disclosure, the 2D image set may be composed of 2D images in which the camera 205 of the 3D scanner 200 and the object 20 maintain the same positional relationship in space, but which are acquired by differently controlling the light source 204 influencing the state of a scanned image. For example, the 2D image set may be composed of at least one 2D image generated when the 3D scanner 200 differently controls the color of the light source 204, the presence or absence of a pattern of light emitted by the light source 204, the interval or type of patterns of light emitted by the light source 204, etc., while looking at the object 20 from the same viewpoint through the camera 205.

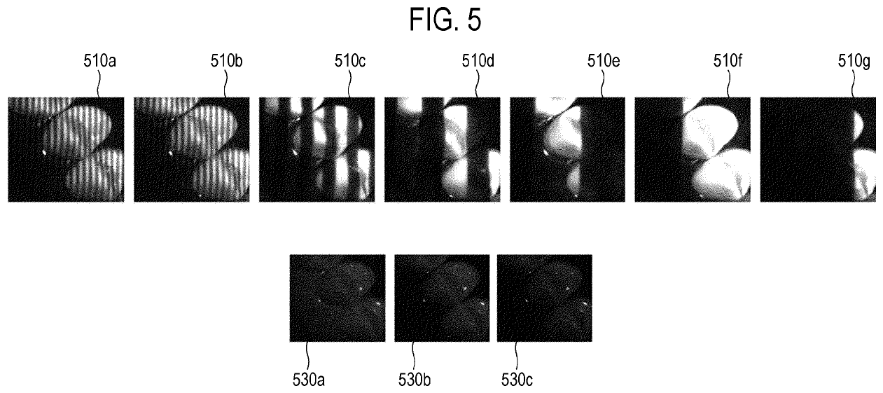

FIG. 5 is an exemplary view showing images included in a 2D image set according to an embodiment of the present disclosure.

The 2D image set may include at least one 2D image acquired by irradiating an object with light with a pattern through the 3D scanner, and at least one 2D image acquired by irradiating an object with light without a pattern through the 3D scanner. Hereinafter, for convenience of explanation, “the 2D image acquired by irradiating an object with light with a pattern” may be simply called a patterned image, and “the 2D image acquired by irradiating an object with light without a pattern” may be simply called a non-patterned image. Patterned images 510a to 510g may be acquired when the 3D scanner 200 irradiates the object 20 with light with a predetermined pattern and captures the patterned light reflected from the object. The patterned images 510a to 510g may be distinguished from each other according to a pattern with which the 3D scanner 200 irradiates the object. For example, the patterned images 510a to 510g may be distinguished from each other according to the shape of the pattern, the interval between patterns, the contrast ratio within the pattern, etc. Non-patterned images 530a to 530c may be acquired when the 3D scanner 200 irradiates the object 20 with light without a pattern and captures the light reflected from the object. The non-patterned images 530a to 530c may be distinguished from each other according to the wavelength and/or color of light emitted from the 3D scanner 200 toward the object. For example, the non-patterned images 530a to 530c may be distinguished from each other according to the color of the emitted light such as red, green, blue, etc. In the present disclosure, the patterned image may include depth information and shape information to be used when the processor 101 generates the 3D scan data of the object. Further, the non-patterned image may include color information to be used when the processor 101 generates the 3D scan data of the object. As described above, in the present disclosure, by generating the 3D scan data based on the 2D image set including at least one patterned image and at least one non-patterned image, the 3D scan data of the object can be generated from a plurality of captured 2D images of the object.

In step S420, the processor 101 may input an input image to an artificial neural network based on the 2D image set. The input image input to the artificial neural network may be generated based on the 2D image set.

In one embodiment of the present disclosure, the input image input to the artificial neural network may be generated based on at least one 2D image acquired by radiating the object with the non-patterned light. Referring to FIG. 5, the input image input to the artificial neural network may be generated based on at least one of, for example, the 2D image 530a acquired by irradiating the object with red light without a pattern, the 2D image 530b acquired by irradiating the object with green light without a pattern, and the 2D image 530c acquired by radiating the object with blue light without a pattern. The processor 101 may detect a region well that is better detected in light of a specific wavelength range through the artificial neural network, by using the non-patterned image, which is acquired by irradiating the object with monochromatic light without a pattern, as the input image input to the artificial neural network. In an additional embodiment of the present disclosure, the processor 101 may generate the input image input to the artificial neural network from a 2D image acquired when the 3D scanner 200 irradiates the object with white light without a pattern. Here, the white light with which the 3D scanner 200 irradiates the object may be light emitted as a result of mixing red light, green light, and blue light.

In one embodiment of the present disclosure, the input image input to the artificial neural network may be an RGB image. At this time, in order to input the input image to the artificial neural network, the processor 101 may generate an RGB image by using two or more 2D images included in a 2D image set and used to acquire monochrome information, and may input the generated RGB image to the artificial neural network. The processor 101 may generate a single RGB image by merging the 2D image 530a acquired by radiating the object with red light without a pattern, the 2D image 530b acquired by radiating the object with green light without a pattern, and the 2D image 530c acquired by irradiating the object with blue light without a pattern, and may input the single RGB image to the artificial neural network. For example, each pixel of a 2D image according to monochromatic light may have one scalar value according to the brightness or intensity of the monochromatic light. In this case, the processor 101 may generate an RGB value (RGB vector) of the corresponding pixel through the scalar value of the monochromatic light for each pixel. Specifically, if the values of pixels at specific positions in the non-patterned images 530a to 530c is 210 in the 2D image 530a, 112 in the 2D image 530b, and 0 in the 2D image 530c, respectively, the processor 101 may determine RGB values of the pixels at the specific positions as (210, 112, 0). The processor 101 may generate an RGB image by using two or more 2D images for obtaining the monochrome information in the same manner as above. The processor 101 may input the generated RGB image to the artificial neural network and detect a first region in the input image. In an additional embodiment of the present disclosure, when the 3D scanner 200 radiates the object with white light without a pattern and the processor 101 acquires a 2D image accordingly, the 2D image may be an RGB image.

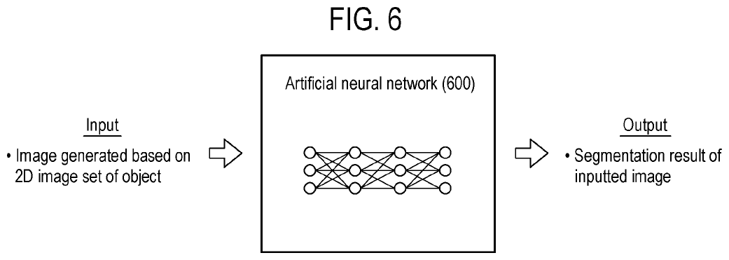

FIG. 6 is a conceptual diagram showing input/output data of an artificial neural network according to an embodiment of the present disclosure.

The artificial neural network 600 of the present disclosure may be an artificial neural network that has been trained to detect at least one predetermined region in an image of an object. Here, the “image of an object” received by the artificial neural network may be an image generated from the 2D image set of the object generated by the 3D scanner, as described above.

The artificial neural network 600 according to one embodiment of the present disclosure may have been trained to output a result of segmentation of an input image by classifying at least one pixel included in the input image into a corresponding region among at least one predetermined region. In the present disclosure, the at least one predetermined region classified through the artificial neural network 600 may include, for example, gingiva, teeth, metal, tongue, cheek, lip, diagnosis model, and the like. The artificial neural network 600 of the present disclosure may have been trained based on one or more learning images labeled with a number of a region corresponding to each pixel of an image. The artificial neural network 600 may have been trained by receiving a learning image, outputting a corresponding region for each pixel, comparing the corresponding region with labeled data, and updating a node weight in the way of backpropagating an error according to the comparison result. A plurality of node weights, bias values, parameters, or the like included in the artificial neural network 600 may have been trained by the processor 101 within the electronic apparatus 100, and may be transmitted to the electronic apparatus 100 after being trained in an external apparatus for use by the processor 101. The processor 101 may input an input image to the trained artificial neural network 600, and may acquire the result of segmentation obtained by classifying at least one pixel included in the input image into a corresponding region among at least one predetermined region, from the artificial neural network 600.

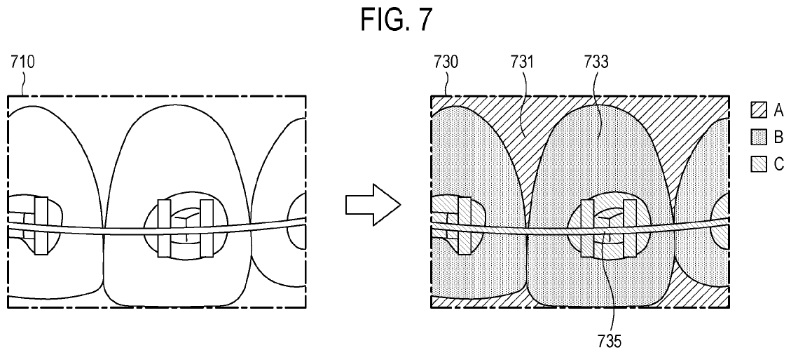

FIG. 7 is a view illustrating input/output data of the artificial neural network according to an embodiment of the present disclosure.

The artificial neural network 600 may receive an input image 710 and output a segmentation result 730 obtained by classifying each pixel into at least one predetermined region. Reference number 730 denotes a visually expressed segmentation result. The segmentation result, which is output data of the artificial neural network, may include at least one predetermined region. For example, the segmentation result 730 may include region A 731, region B 733, and region C 735 according to the corresponding regions, and the region A, the region B, and the region C in the input image 710 may be regions corresponding to gingiva, teeth, and metal, respectively.

In step S430, the processor 101 may detect the first region in the input image based on the output of the artificial neural network. The first region detected by the processor 101 according to one embodiment of the present disclosure based on the output of the artificial neural network may be a region corresponding to metal on an image of the object. Specifically, the region corresponding to the metal may be, for example, a region corresponding to a wire, a prosthesis, or the like for orthodontic treatment on the image of the object. For example, when the region detected by the processor 101 as the first region is a region corresponding to metal, the processor 101 may detect the region C 735 as the first region in the segmentation result 730 that is the output of the artificial neural network. In the present disclosure, the first region may be a region to be excluded from the 3D scan data of the object, in which case, the first region may be interchangeably called an “exclusion target region.”

In step S440, the processor 101 may generate 3D scan data of the object based on the first region. The processor 101 according to one embodiment of the present disclosure may generate the 3D scan data based on the 2D image set such that coordinates corresponding to the first region are not included in the 3D scan data. Specifically, when generating the 3D scan data based on the 2D image set, the processor 101 according to one embodiment of the present disclosure may generate the 3D scan data excluding the values of pixels corresponding to the first region in each 2D image included in the 2D image set from a calculation target. For example, a plurality of 2D images included in the 2D image set may be images obtained by photographing the same object with light having different patterns or colors. In this case, the plurality of 2D images may share the same reference coordinate system (e.g., the 2D coordinate system). Accordingly, the processor 101 may determine a region at the same position as the first region detected by the artificial neural network even within each 2D image included in the 2D image set. As a result, the processor 101 may generate the 3D scan data excluding the exclusion target region by generating the 3D scan data excluding the values of pixels corresponding to the first region in each 2D image included in the 2D image set.

In order to generate the 3D scan data about the object based on the first region, the processor 101 according to one embodiment of the present disclosure may remove data of a region corresponding to the first region in at least one 2D image included in the 2D image set. For example, the processor 101 may change the values of pixels included in the region corresponding to the first region in at least one 2D image included in the 2D image set to a preset value. The preset value may be referred to as a default value, and may be, for example, a real number such as “−1” or “0”. The processor 101 may remove information indicated by the values of pixels corresponding to the first region by changing the values of pixels corresponding to the first region in the 2D image to the preset value. By changing the values of pixels corresponding to the first region to the preset value, the processor 101 may exclude the corresponding pixels on the 2D image from a calculation target when generating the 3D image.

The processor 101 according to one embodiment of the present disclosure may generate the 3D scan data using at least one 2D image from which the data of the region corresponding to the first region are removed. The processor 101 may exclude from the calculation target a region to be excluded in advance before generating the 3D scan data by detecting the first region to be excluded (e.g., a metal region) in the 2D image based on the 2D image set acquired from the 3D scanner 200 and removing the first region from the 2D image according to the detection result. After that, the processor 101 may display the generated 3D scan data on the display 107. In this way, unlike a method of removing an exclusion target region from 3D scan data generated after the 3D scan data are generated, by performing a method of acquiring the 2D image and at the same time removing an exclusion target region from the 2D image in real time in the step of acquiring the 2D image, and generating the 3D scan data based on at least one 2D image from which the exclusion target region has been removed, the processor 101 according to the present disclosure may reduce a computational burden and increase the overall computational speed by reducing the size of calculation target data.

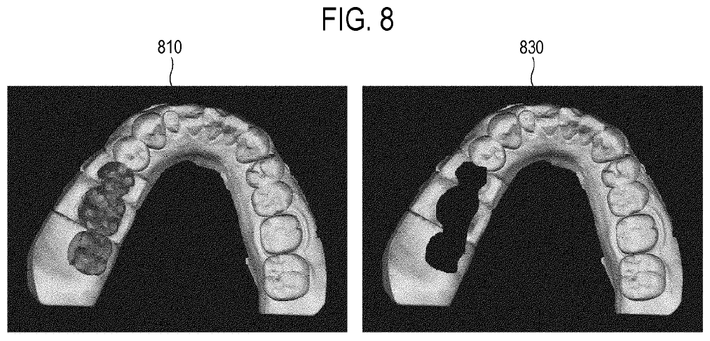

FIG. 8 is a view illustrating 3D scan data according to an embodiment of the present disclosure. Reference numerals 810 and 830 denote cases where a region corresponding to metal is included in the 3D scan data of an object and is excluded from the 3D scan data of the object, respectively. When generating the 3D scan data of the object based on the first region, since the coordinates corresponding to the first region cannot be included in the 3D scan data, the processor 101 according to the present disclosure can prevent a region, which is not desired by a user, from being expressed within the 3D scan data. Through this, the present disclosure can exclude unnecessary information for examination or treatment from scan data and provide necessary information (patient's unique oral cavity information) to the user more accurately and concisely.

The processor 101 according to an embodiment of the present disclosure may detect a plurality of different first regions in the input image based on the output of the artificial neural network 600 and generate 3D scan data so that the plurality of detected first regions are distinguished from each other. In order to distinguish the plurality of first regions from each other within the 3D scan data, the processor 101 may, for example, label pixels corresponding to each detected first region with different numbers in the 3D scan data.

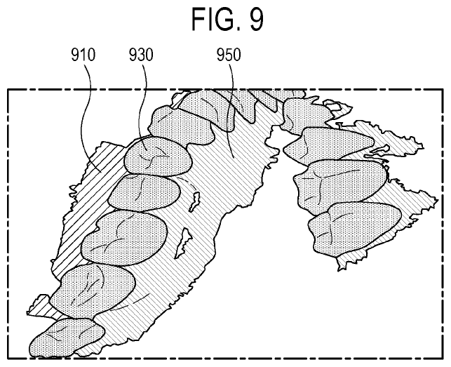

FIG. 9 is an exemplary view visually representing the 3D scan data generated to distinguish the plurality of first regions from each other according to an embodiment of the present disclosure. The 3D scan data shown in FIG. 9 includes a cheek inside region 910, a tooth region 930, and a gingival region 950 as the plurality of first regions. The processor 101 may generate the 3D scan data in which the plurality of first regions are distinguished from each other, by generating an input image to be input to an artificial neural network based on a 2D image set acquired through a 3D scanner and acquiring a segmentation result by inputting the generated input image to the artificial neural network. When the plurality of first regions included in the 3D scan data are visually divided and provided to a user, the user can quickly and conveniently distinguish between the plurality of different first regions included in the 3D scan data.

The processor 101 according to an embodiment of the present disclosure may acquire user input regarding whether or the first region is included, from the user through the input device 109. When generating the 3D scan data, the processor 101 may determine whether or not the coordinates corresponding to the first region are included in the 3D scan data according to the acquired user input.

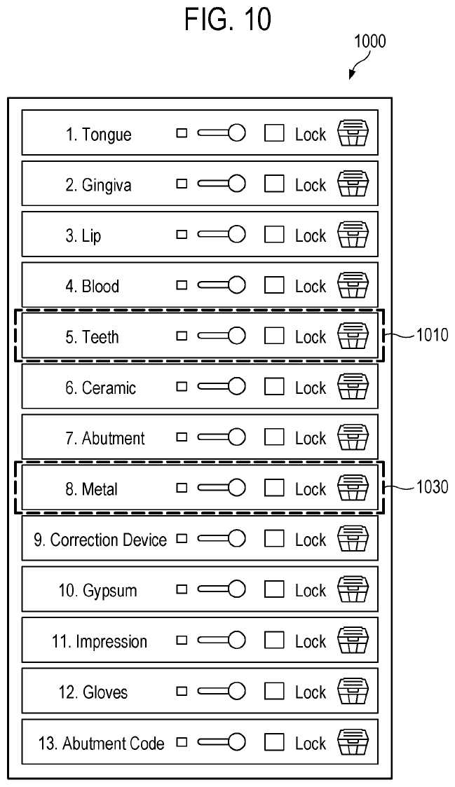

FIG. 10 is an exemplary diagram showing a user interface screen for receiving user input regarding whether or not a specific region is included in the 3D scan data. The user interface screen 1000 of FIG. 10 may be provided to a user through the display 107 of the electronic apparatus 100. The user may transmit the user input to the electronic apparatus 100 by touching a predetermined region of the user interface screen 1000. As one example, the user may transmit, to the electronic apparatus 100 through a sub-screen 1010 related to “teeth” in the user interface screen 1000, the user input on whether or not a “tooth” region is included in the 3D scan data to be generated by the processor 101. For example, the user may prevent (i.e., turn off) the “tooth” region from being included in the 3D scan data by touching the sub-screen 1010 related to “teeth.” When no user input is received for the sub-screen 1010 related to “teeth” (i.e., in a default state), the processor 101 may be configured to include the “tooth” region in the 3D scan data. As another example, the user may transmit, to the electronic apparatus 100 through a sub-screen 1030 related to “metal” in the user interface screen 1000, the user input regarding whether a “metal” region is included in the 3D scan data.

In one embodiment of the present disclosure, the processor 101 may acquire user input regarding whether or not each of a plurality of first regions is included, through the user interface screen 1000. The processor 101 may provide a user with a plurality of sub-screens through which it can be determined whether each of the plurality of first regions is included. Through manipulation of each of the plurality of sub-screens, the user may distinguish between regions to be expressed and regions to be excluded on the 3D scan data among the plurality of first regions such as a tongue, lips, teeth, metal, etc., and the scan data may be generated accordingly. Further, among metals, the user may distinguish between a first region corresponding to an orthodontic device and a first region corresponding to a prosthetic device.

According to the present disclosure in various embodiments, since a specific region is detected in a scanned image using an artificial neural network and 3D scan data related to an object are generated based on the detected specific region, when a user wants to generate 3D scan data that does not include the specific region, the specific region may not be expressed within the 3D scan data. Through this, the present disclosure can exclude unnecessary information for examination or treatment from the 3D scan data and provide necessary information to the user more accurately and concisely.

According to the present disclosure in various embodiments, when a plurality of first regions included in the 3D scan data are visually divided and provided to a user, the user can quickly and conveniently distinguish between the plurality of different first regions included in the 3D scan data.

While the foregoing methods have been described with respect to particular embodiments, these methods may also be implemented as computer-readable code on a computer-readable recording medium. The computer-readable recording medium includes any kind of data storage devices that can be read by a computer system. Examples of the computer-readable recording medium includes ROM, RAM, CD-ROM, magnetic tape, floppy disk, optical data storage device and the like. Also, the computer-readable recording medium can be distributed to computer systems which are connected through a network so that the computer-readable code can be stored and executed in a distributed manner. Further, the functional programs, code, and code segments for implementing the foregoing embodiments can easily be inferred by programmers in the art to which the present disclosure pertains.

While certain embodiments have been described, these embodiments have been presented by way of example only, and are not intended to limit the scope of the disclosures. Indeed, the embodiments described herein may be embodied in a variety of other forms. Furthermore, various omissions, substitutions and changes in the form of the embodiments described herein may be made without departing from the spirit of the disclosures. The accompanying claims and their equivalents are intended to cover such forms or modifications as would fall within the scope and spirit of the disclosures.

What is claimed is:

1. A method of processing scanned images of a 3D scanner performed by an electronic apparatus, comprising:
acquiring, from the 3D scanner, a 2D image set of an object generated by scan of the 3D scanner, the 2D image set including at least one 2D image;
inputting an input image to an artificial neural network, which has been trained to detect at least one predetermined region in an image of the object, based on the 2D image set;
detecting a first region in the input image based on an output of the artificial neural network; and
generating 3D scan data of the object based on the first region.

2. The method of claim 1, wherein the first region is a region corresponding to metal in the input image.

3. The method of claim 1, wherein the input image input to the artificial neural network is generated based on the at least one 2D image acquired by irradiating the object with non-patterned light.

4. The method of claim 1, wherein inputting the input image to the artificial neural network comprises:
generating an RGB image using two or more 2D images, which are included in the 2D image set and used to acquire monochrome information; and
inputting the RGB image to the artificial neural network.

5. The method of claim 1, wherein the artificial neural network has been trained to output a segmentation result for the input image by classifying at least one pixel included in the input image into a corresponding region among the at least one predetermined region.

6. The method of claim 1, wherein generating the 3D scan data of the object comprises:
generating the 3D scan data based on the 2D image set such that coordinates corresponding to the first region are not included in the 3D scan data.

7. The method of claim 1, wherein generating the 3D scan data of the object comprises:
removing data of a region corresponding to the first region from the at least one 2D image included in the 2D image set; and
generating the 3D scan data using the at least one 2D image from which the data of the region corresponding to the first region are removed.

8. The method of claim 7, wherein removing the data of the region corresponding to the first region from the at least one 2D image included in the 2D image set comprises:
changing values of pixels included in the region corresponding to the first region in the at least one 2D image to a preset value.

9. The method of claim 1, wherein detecting the first region comprises:
detecting a plurality of different first regions in the input image based on the output of the artificial neural network, and
wherein generating the 3D scan data comprises:
generating the 3D scan data such that the plurality of first regions are distinguished from each other.

10. The method of claim 1, further comprising:
acquiring user input on whether the first region is to be included,
wherein generating the 3D scan data comprises:
determining whether coordinates corresponding to the first region are included in the 3D scan data according to the user input.

11. An electronic apparatus comprising:
a communication circuit communicatively connected to a 3D scanner;
a memory;
a display; and
one or more processors,
wherein the one or more processors are configured to:
acquire, from the 3D scanner, a 2D image set of an object generated by scan of the 3D scanner, the 2D image set including at least one 2D image;
input an input image to an artificial neural network, which has been trained to detect at least one predetermined region in an image of the object, based on the 2D image set;
detect a first region in the input image based on an output of the artificial neural network; and
generate 3D scan data of the object based on the first region.

12. The electronic apparatus of claim 11, wherein the first region is a region corresponding to metal in the input image.

13. The electronic apparatus of claim 11, wherein the input image input to the artificial neural network is generated based on at least one 2D image acquired by irradiating the object with non-patterned light.

14. The electronic apparatus of claim 11, wherein the one or more processors are configured to:
generate an RGB image using two or more 2D images, which are included in the 2D image set and used to acquire monochrome information; and
input the RGB image to the artificial neural network.

15. The electronic apparatus of claim 11, wherein the artificial neural network has been trained to output a segmentation result for the input image by classifying at least one pixel included in the input image into a corresponding region among the at least one predetermined region.

16. The electronic apparatus of claim 11, wherein the one or more processors are configured to:
generate the 3D scan data based on the 2D image set such that coordinates corresponding to the first region are not included in the 3D scan data.

17. The electronic apparatus of claim 11, wherein the one or more processors are configured to:
remove data of a region corresponding to the first region from the at least one 2D image included in the 2D image set; and
generate the 3D scan data using the at least one 2D image from which the data of the region corresponding to the first region are removed.

18. The electronic apparatus of claim 17, wherein the one or more processors are configured to:
change values of pixels included in the region corresponding to the first region in the at least one 2D image to a preset value.

19. The electronic apparatus of claim 11, wherein the one or more processors are configured to:
detect a plurality of different first regions in the input image based on the output of the artificial neural network; and
generate the 3D scan data so that the plurality of first regions are distinguished from each other.

20. A non-transitory computer-readable recording medium storing instructions that, when executed by one or more processors, cause the one or more processors to perform operations, the instructions causing the one or more processors to:
acquire, from a 3D scanner, a 2D image set of an object generated by scan of the 3D scanner, the 2D image set including at least one 2D image;
input an input image to an artificial neural network, which has been trained to detect at least one predetermined region in an image of the object, based on the 2D image set;
detect a first region in the input image based on an output of the artificial neural network; and
generate 3D scan data of the object based on the first region.
